(19) United States
(12) Patent Application Publication
(10) Pub. No.: US 2004/0042999 A1
(43) Pub. Date: Mar. 4, 2004

(54) Method of improving systemic exposure of subcutaneously administered therapeutic proteins

(76) Inventors: Charles B. Davis, Collegeville, PA (US); Amarnath Sharma, Unite Square (SG)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/415,738

(22) PCT Filed: Nov. 1, 2001
(86) PCT No.: PCT/US01/51617

(51) Int. Cl. A61K 38/20

(52) U.S. Cl. 424/85.2

(57) ABSTRACT

A method of improving systemic exposure of subcutaneously administering therapeutic proteins.

### BACKGROUND OF THE INVENTION

Currently, there are numerous therapeutic proteins in clinical testing or development for a variety of therapeutic applications such as for organ transplantation, treatment of autoimmune disease, restenosis, certain forms of cancer, as well as prophylactic applications, e.g., as an anti-viral agent. Typically such proteins are administered either intravenously (iv) or subcutaneously (sc), although other routes of administration are also possible, e.g., intramuscularly (im) and intranasally. In general, sc administration is preferable over iv administration, for iv administration requires catheterization for administration in a home setting, medical attention when administered in a clinic or physician's office, or hospitalization in more extreme circumstances. In addition, a therapeutic delivered iv takes longer to administer when compared to sc administration, and as a result is a more costly therapy. However, sc administration is not without drawbacks. For example, there are physical limitations on the maximum dose which can be delivered at the injection site.

It has been observed that large polypeptides when administered subcutaneously, are first absorbed into the lymphatic system from the site of injection and then subsequently migrate into the blood stream (see, e.g., Weinstein et al., Science, 222: 423-426 (1983), Weinstein et al., Cancer Invest., 3:85-95 (1985), Supersaxo et al., Pharm. Res., 7:167-169 (1990)). For therapeutic targets not located in the lymphatic system, e.g., respiratory syncytial virus (RSV), the systemic exposure of, e.g., an anti-RSV monoclonal antibody administered sc, is comparable to that administered iv and the bioavailabihty is not affected by the amount of the therapeutic protein administered (see, e.g., Davis et al., Drug Met. Disp., 23:1028-1036 (1995)).

When there are target receptors and/or endogenous binding protein(s) present in the lymphatic system, the extent of absorption (i.e., systemic exposure) is affected by binding of therapeutic proteins to such targets in the lymphatic system, thus influencing the amount of the therapeutic protein that enters the blood stream (see e.g., Davis and Bugelski, Drug Delivery, 5: 95-100 (1998)). To treat systemic diseases, such as cancer, it is desirable for the therapeutic protein to reach the site(s) of action in an effective amount. For a subcutaneous route of administration, it is essential for the therapeutic protein to enter the blood stream and not remain in the lymph nodes or other regions of the lymphatic system.

Hence, the need exists to effectively deliver therapeutic proteins, to treat systemic diseases. The methods described herein will become apparent to those of ordinary skill in the art upon reading this specification.

### SUMMARY OF THE INVENTION

The present invention relates generally to the field of therapeutic proteins, and dosing regimens that enhance systemic exposure and thus pharmacologic effectiveness of therapy. The present invention provides an improved method for treating diseases by increasing the systemic response to a therapeutic protein which binds to specific receptors and/or endogenous proteins present in the lymphatic system. More specifically, the present invention provides an improved method for treating diseases by increasing the systemic exposure to interleukin-18 (EL-18) which binds to specific receptors and/or endogenous proteins present in the lymphatic system.

### BRIEF DESCRIPTION OF THE DRAWINGS

FIG. 1—FIG. 1 is a graph showing mean plasma IL-18 concentration versus time profiles following an intravenous dose (0.1, 1 or 10 mg/kg) to Cynomolgus monkeys.

FIG. 2(a)—FIG. 2a is a graph showing mean plasma IL-18 concentrations versus time profiles following single and repeat intravenous administration (10 mg/kg/day for 5 days).

FIG. 2(b)—FIG. 2b is a graph showing mean plasma EL-18 concentrations versus time profiles following single and repeat intravenous administration (30 mg/kg/day for 5 days).

FIG. 2(c)—FIG. 2c is a graph showing mean plasma IL-18 concentrations versus time profiles following single and repeat intravenous administration (75 mg/kg/day for 5 days).

FIG. 3(a)—FIG. 3a is a graph showing mean plasma IL-18 concentration versus time profiles following single and repeat subcutaneous administration (10 mg/kg/day for 4 days).

FIG. 3(b)—FIG. 3b is a graph showing mean plasma IL-18 concentration versus time profiles following single and repeat intravenous administration (10 mg/k-g/day for 5 days).

FIG. 4—FIG. 4 is a graph showing mean (SD) bioavailability of IL-18 following single and repeat subcutaneous administration.

### DETAILED DESCRIPTION OF THE INVENTION

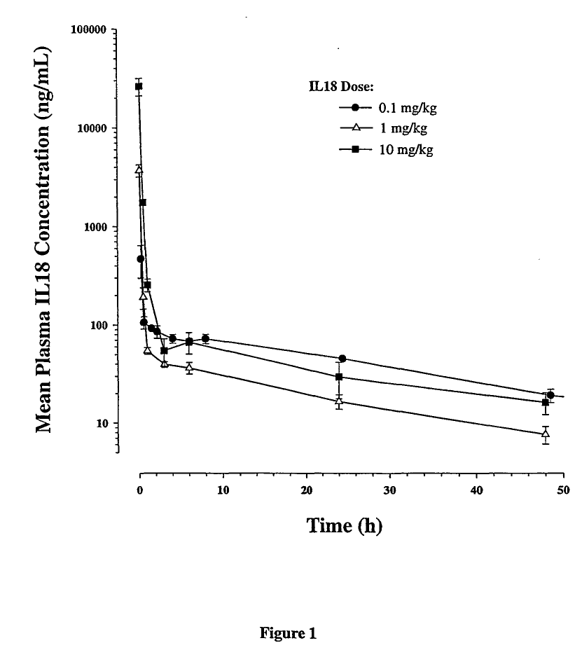

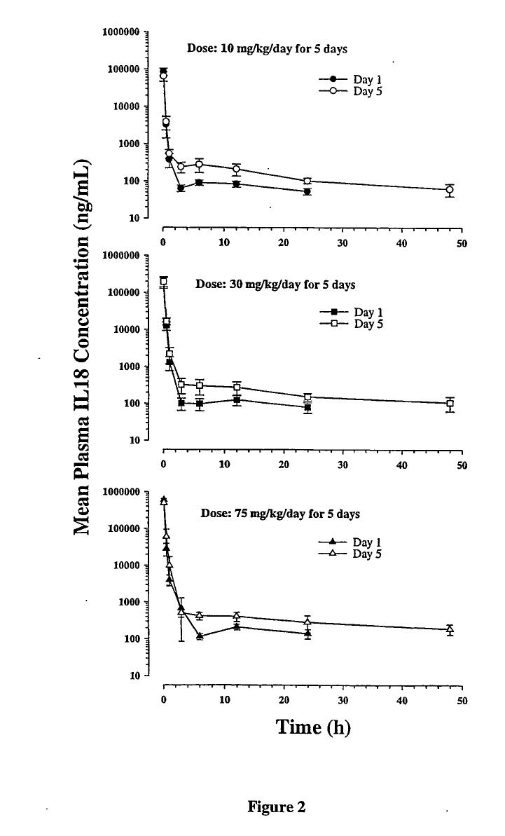

The Applicants have discovered that multiple subcutaneous administrations of therapeutic proteins, such as IL-18, result in unexpected accumulation of the protein therapeutic. By employing this method, the systemic exposure to a therapeutic protein, such as IL-18, is increased more than expected based on the single subcutaneous dose pharmacokinetic profile and more than expected based on the single-and multiple intravenous dose pharmacokinetic profile and allows for the improved treatment of systemic diseases, such as cancer, bacterial infections, viral infections, fungal infections and parasitic infections. By first providing (or administering) a saturating subcutaneous dose (or doses) of a therapeutic protein, such as IL-18, subsequent administrations, also given subcutaneously, result in at least 50% greater systemic exposure than an equivalent subcutaneous dose administered without the benefit of the saturating dose or doses. Preferably, the systemic exposure of such therapeutic protein is increased by at least 2-fold, more preferably it is increased by at least 4fold. The relative systemic exposure of a single sc dose compared to a single iv dose, i.e., the apparent absolute bioavailability, is approximately 15% in Cynomolgus monkeys (compare Tables 1 and 2, day 1 AUC₀₋₂₄, 10 mg/kg). FIG. 1 shows mean plasma IL-18 concentration versus time profiles following a single intravenous dose (0.1, 1 or 10 mg/kg) to monkeys. Plasma concentrations declined in a bi-phasic manner after intravenous administration with a steep initial phase characterized by a half-life of ˜5 min. The terminal disposition phase had a long half-life of ˜24 h but the concentrations during this phase were low. Due to a relatively rapid clearance from blood following iv administration, iv administered IL-18 does not accumulate with multiple dosing, and when IL-18 is administered daily, the systemic exposure over 24 h (AUC₀₋₂₄) is approximately the same from day to day (Table 1, day 1 vs day 5 AUC₀₋₂₄). This applies to iv administration over a broad range of doses (Table 1, 10-75 mg/kg per day). FIGS. 2a-2c show mean plasma IL-18 concentraton versus time profiles following single and repeat intravenous administration (10-75 mg/kg). The daily systemic exposure increased in a dose proportional manner between doses of 10 and 75 mg/kg following single and repeat iv dosing, indicating linear pharmacokinetics of IL-18(see also Table 1). There was no marked change in the maximum plasma IL-18 concentration following 5 daily intravenous doses, as Cₘₐₓ values were similar on both Day 1 and Day 5 (Table 1).

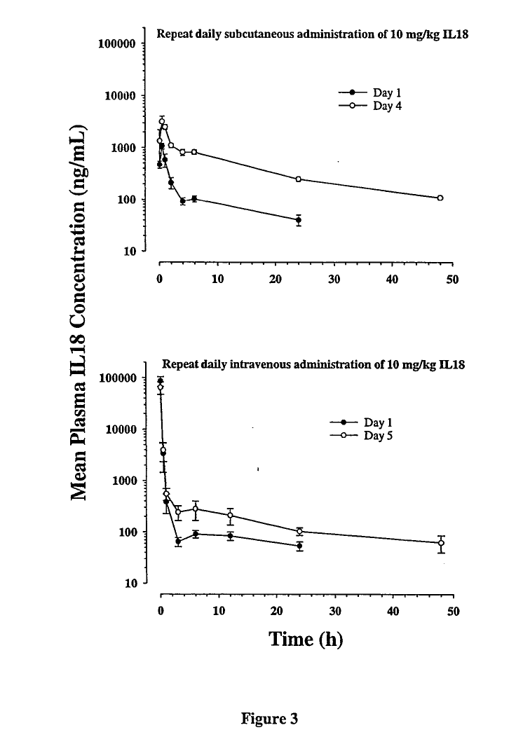

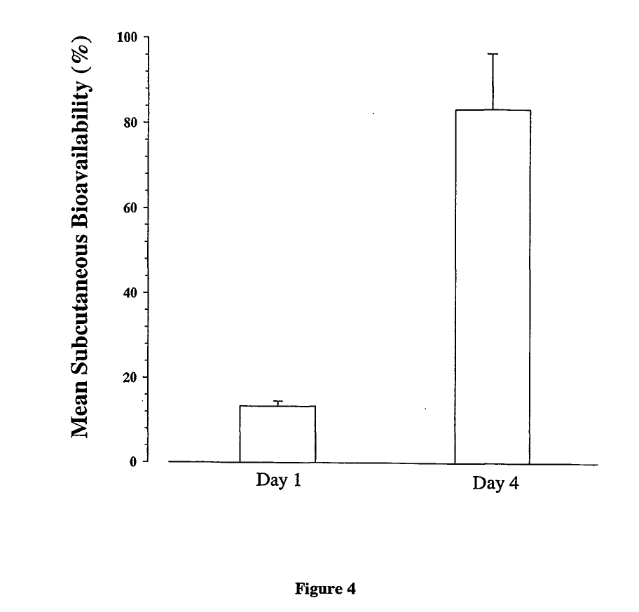

However, the systemic exposure (AUC₀₋₂₄) to IL-18 in the Cynomolgus monkey following daily sc administration increases over time so that after 4 days of administration, the systemic exposure from the 4th sc administration is comparable to the systemic exposure following an iv dose of the same amount. FIGS. 3a and 3b show the mean plasma IL-18 concentration versus time profiles following single and repeat subcutaneous or intravenous administration (10 mg/cg dose). After subcutaneous administration, the maximum plasma concentrations were observed at −0.5 hours indicating that IL-18 is rapidly absorbed from the sc injection site. In contrast to the lack of accumulation following 5 daily intravenous doses of 10 mg/kg, 4 daily subcutaneous doses of 10 mg/kg IL-18 to cynomolgus monkeys resulted in significant accumulation (see also Table 2). Specifically, the apparent absolute bioavailability was increased to approximately 100% (FIG. 4; compare Tables 1 and 2, day 4 sc AUC₀₋₂₄, relative to day 1 iv AUC₀₋₂₄, 10 mg/kg). This is theoretically the maximum bioavailability that can be obtained following extravascular administration for any protein therapeutic. There was also a marked change in the maximum plasma IL-18 concentration following 4 daily sc doses, as Cₘₐₓ values were 3-fold higher on day 4 compared with day 1 (Table 2). Similar phenomena were observed in Rhesus monkeys following 7 daily sc administrations of IL-18 at lower doses (Table 3, 0.1-1 mg/kg/day).

TABLE 1
Intravenous Pharmacokinetics of IL-18 in Cynomolgus monkeys

| | Males (n = 2) | | | | Females (n = 2) | | | |
|---|---|---|---|---|---|---|---|---|
| IV | Cₘₐₓ | | AUC₀₋₂₄ | | Cₘₐₓ | | AUC₀₋₂₄ | |
| group | (ug/mL) | | (ug.h/mL) | | (ug/mL) | | (ug.h/mL) | |
| Dose | Day | Day | Day | Day | Day | Day | Day | Day |
| (mg/kg) | 1 | 5 | 1 | 5 | 1 | 5 | 1 | 5 |
| 10 | 82.0 | 61.0 | 20.0 | 17.4 | 89.8 | 68.6 | 21.4 | 22.3 |
| 30 | 223 | 219 | 50.3 | 61.0 | 165 | 175 | 42.7 | 55.8 |
| 75 | 611 | 526 | 150 | 165 | 591 | 472 | 133 | 142 |

TABLE 2
Subcutaneous Pharmacokinetics of IL-18 in Cynomolgus monkeys

| SC | Males (n = 2) | | | | Females (n = 2) | | | |
|---|---|---|---|---|---|---|---|---|
| group | Cₘₐₓ | | AUC₀₋₂₄ | | Cₘₐₓ | | AUC₀₋₂₄ | |
| Dose | (ug/mL) | | (ug.h/mL) | | (ug/mL) | | (ug.h/mL) | |
| (mg/ | Day | Day | Day | Day | Day | Day | Day | Day |
| kg) | 1 | 4 | 1 | 4 | 1 | 4 | 1 | 4 |
| 10 | 1.13 | 3.45 | 2.93 | 16.8 | 0.999 | 2.93 | 2.62 | 15.7 |

TABLE 3
Subcutaneous Pharmacokinetics of IL-18 in Rhesus monkeys

| | Males (n = 2) | | | | Females (n = 2) | | | |
|---|---|---|---|---|---|---|---|---|
| | Cₘₐₓ | | AUC₀₋₂₄ | | Cₘₐₓ | | AUC₀₋₂₄ | |
| | (ug/mL) | | (ug.h/mL) | | (ug/mL) | | (ug.h/mL) | |
| SC Dose | Day | Day | Day | Day | Day | Day | Day | Day |
| (mg/kg) | 1 | 7 | 1 | 7 | 1 | 7 | 1 | 7 |
| 0.1 | 0.060 | 0.069 | 0.418 | 0.977 | 0.073 | 0.067 | 0.465 | 0.966 |
| 0.3 | 0.072 | 0.140 | 0.520 | 1.608 | 0.079 | 0.185 | 0.558 | 2.247 |
| 1.0 | 0.446 | 0.301 | 1.156 | 2.898 | 0.222 | 0.339 | 0.924 | 3.320 |

We claim:

1. A method for improving treatment of a systemic disease with a therapeutic protein comprising:
(a) administration of a first saturating subcutaneous dose of the therapeutic protein to a patient in need thereof; and
(b) administration of one or more subsequent subcutaneous doses of the therapeutic protein to the patient;
wherein the systemic exposure of said therapeutic protein from the second or subsequent administrations is at least 50% greater than the first and equivalent subcutaneous dose of the therapeutic protein.

2. The method of claim 1 wherein the therapeutic protein is IL-18.

3. The method of claim 2 wherein the systemic disease is cancer.

4. The method of claim 2 wherein the systemic disease is a bacterial infection.

5. The method of claim 2 wherein the systemic disease is a viral infection.

6. The method of claim 2 wherein the systemic disease is a fungal infection.

7. The method of claim 2 wherein the systemic disease is a parasitic infection.

8. The method of claim 1 wherein the therapeutic protein is IL-18 conjugated to a water-soluble polymer.

9. The method of claim 1 wherein the systemic exposure of said therapeutic protein from the second or subsequent administrations is at least 100% greater than the first and equivalent subcutaneous dose of the therapeutic protein.

10. The method of claim 1 wherein the systemic exposure of said therapeutic protein from the second or subsequent administrations is comparable to the systemic exposure following an equivalent single intravenous administration of the therapeutic protein.
